(19) United States
(12) Patent Application Publication
(10) Pub. No.: US 2008/0019861 A1
(43) Pub. Date: Jan. 24, 2008

(54) Air Treatment Method and Device

(76) Inventor: Hermannus Gerhardus Maria Silderhuis, Enschede (NL)

Correspondence Address: THE WEBB LAW FIRM, P.C., 700 KOPPERS BUILDING, 436 SEVENTH AVENUE, PITTSBURGH, PA, 15219, US

(21) Appl. No.: 10/572,082

(22) PCT Filed: Oct. 26, 2004
(86) PCT No.: PCT/NL04/00752

§ 371 Date: Nov. 1, 2006

(30) Foreign Application Priority Data
Oct. 27, 2003 (NL) 03/00730
Mar. 26, 2004 (NL) 04/000209

(51) Int. Cl. A61L 9/16; A61L 9/20; A61L 9/22; F24F 3/16

(52) U.S. Cl. 422/3; 422/4; 96/134; 96/224

(57) ABSTRACT

To improve the air quality in bounded spaces such as a room, an air treatment device and an air treatment method are disclosed. The air treatment device comprises a fan for stimulating airflow through the air treatment device and an UV treatment chamber. An UV radiation source radiates UV radiation in the UV treatment chamber to kill microorganisms in said airflow. The air treatment device is designed such that a high airflow may be generated, while all microorganisms present in the air flowing through the air treatment device are killed. With the high airflow and air cleaning capacity, the air treatment device may clean a bounded space in a short period of time.

The present invention relates to an air treatment method and an air treatment device for killing microorganisms present in air.

In bounded spaces, such as rooms, in houses, buildings or other human or animal living environments, numerous pollutants such as dust and microorganisms like viruses, bacteria and fungae are present. These pollutants endanger the health of the human beings or animals living in these bounded spaces.

Air treatment devices for improving the air quality in bounded spaces are known, e.g. from U.S. Pat. No. 5,185,015. The known air treatment device comprises three filters. A first filter filters particles being greater than a predetermined size from the air, a second filter filters particles of selected chemical species and a third filter removes the capacity of airborne bacteria to reproduce by irradiating ultraviolet light.

The known air treatment device however has a limited air cleaning capacity, and has a limited airflow capacity. Having a small airflow capacity the air treatment device is only effective if it is used in a small room that is kept closed over a long period of time. After the room is exposed to normal, polluted air, for example when a door or window is opened, the room is contaminated again and it takes a long period of time again to decontaminate the air in the room, which has to be closed again for this purpose.

Moreover, the known air treatment device is only suited for removing relatively large microorganisms from the air. The known air treatment device uses conventional filters for removing particles having a diameter larger than a predetermined filter diameter. Microorganisms having a smaller diameter may pass the filters and thus remain in the air.

Increasing the airflow capacity of the air treatment device is only possible if all bacteria and other microorganisms such as viruses are completely destroyed. If ultraviolet light is used in doses that will not kill microorganisms, microorganisms get mutated, since microorganisms only get killed after receiving certain doses of ultraviolet light. Since mutated microorganisms may form even a greater threat to humans and animals than non-mutated microorganisms, the microorganisms need to receive at least that certain minimum doses of ultraviolet light to ensure that they get killed. A high capacity air treatment device therefore needs to be designed and configured to ensure that all microorganisms get killed and no mutated microorganisms leave the air treatment device.

It is an object of the present invention to provide an air treatment device that is suited for killing small microorganisms.

The above object is achieved in an air treatment device comprising:
a housing comprising an air inlet and an air outlet;
a fan for stimulating an airflow through the housing from the air inlet to the air outlet; and
an UV treatment chamber downstream relative to the air inlet, said UV treatment filter comprising at least one UV radiation source for exposing said airflow to UV radiation for killing a microorganism present in said airflow.

The air treatment device according to the present invention is configured to expose microorganisms present in air to UV radiation in order to kill said microorganisms instead of removing microorganisms using one or more conventional filters. Thus, the air treatment device is suited for killing a microorganism of any size instead of only a microorganism having a size larger than a predetermined filter diameter.

Large microorganisms need a large dose of UV radiation to get killed, while small microorganisms only need a relatively small dose. Therefore, the air treatment device may comprise at least one filter upstream relative to the UV treatment chamber for removing particles and microorganisms having a size larger than a predetermined filter diameter from said airflow before exposing said airflow to said UV radiation. Thus, only small microorganism reach the UV treatment chamber. Said small microorganisms may be killed by a small dose of UV radiation, thus requiring less UV radiation for killing all microorganisms.

In the UV treatment chamber, the air in the airflow, and in particular each microorganism in the air, is irradiated by UV radiation. Each microorganism is to receive the above-mentioned minimum dose of UV radiation to be killed. This means that each microorganism is to receive a certain power of UV radiation during a certain period of time. Thereto the UV treatment chamber is configured such that the air remains in the UV treatment chamber during a predetermined minimum period of time and the at least one UV radiation source emits a predetermined UV power.

A suitable UV radiation source emits UV radiation with a wavelength of about 253-257 nm, in particular with a wavelength of 253.7 nm.

To decontaminate large amounts of air per unit time, all elements in the air treatment device, in particular the filters, may be complementary selected and positioned relative to each other. In an embodiment, the air treatment device according to the present invention may comprise a dust filter and a HEPA filter. The dust filter removes all large particles such as dust particles from the air flowing through the housing. Preferably the dust filter is a removable and/or washable filter to be able to easily clean the filter and to have a long use life of the dust filter.

Smaller particles that are not removed by the dust filter may be removed by the HEPA (high efficiency particle arrestance) filter. An HEPA filter is a filter type known in the art to remove small particles. A range of HEPA filters is known, the filters in said range differing in the percentage of particles larger than 0.3 micron that is removed by said filter.

In the embodiment according to the present invention, an HEPA filter constructed of glass fiber and removing about 99.97% of the particles larger than 0.3 micron is preferably used. Such an HEPA filter is known as a H13 HEPA filter and removes about all dust particles and also removes large bacteria from the air.

Instead of a dust filter and/or a HEPA filter, any other filter may be employed for removing pollutants having a size larger than a predetermined size. For example, a carbon filter may be employed.

As mentioned above, a filter, e.g. a HEPA filter, may remove large bacteria from the air. These large bacteria thus remain in the filter. Since the filter functions as a hothouse, a large bacteria growth is to be expected, which may result in mutated bacteria. Further, the filter wears off in the course of time due to the air and particles flowing through the filter. Therefore, in the course of time, larger particles and in particular larger bacteria, even the ones earlier caught in the filter, may flow through the HEPA filter. To avoid these effects, a filter UV radiation source radiates UV radiation on the filter to kill the bacteria that remain on the filter. A suitable filter UV radiation source emits UV radiation with a wavelength of about 253-257 nm, in particular with a wavelength of 253.7 nm.

Thus, by killing the bacteria caught by the filter, no bacteria, which may have grown in population and/or may have mutated during their stay on the filter, may flow through the filter in the course of time. Further, the filter may be safely replaced by a new filter as soon as the filter has worn off without having to take the old filter out with a large amount of possibly mutated bacteria thereon.

To kill bacteria, the bacteria need to receive a certain minimum dose of UV radiation. The received dose of UV radiation is equal to the UV power times the time during which the bacteria are exposed to said UV power. Thus, using a high-power UV radiation source, the bacteria need to be exposed only during a short period of time to get killed. However, the bacteria caught on the filter cannot move. Therefore, the filter UV radiation source may be a low-power UV radiation source, since the bacteria may be exposed during a long time, in the end resulting in receiving the required minimum dose to get killed.

To ensure that all microorganisms receive UV radiation in the UV treatment chamber and no microorganisms may pass the at least one UV radiation source in the shadow of other microorganisms, the fan may be positioned in the air treatment device such that the airflow in the UV treatment chamber is turbulent. This means that the fan may be positioned upstream relative to the UV treatment chamber, since the airflow stimulated by the fan is always turbulent at the pressure side of the fan. At the side from where the air is drawn, the airflow may be laminar for relatively low airflow rates. However, it is noted that for high airflow rates, the flow is turbulent at the drawing side and thus in the device according to the present invention the fan may also be positioned downstream of the UV treatment chamber when only using high airflow rates.

An inner wall of the UV treatment chamber may be provided with an UV radiation reflecting layer. UV radiation emitted by the UV radiation source-may thus be more efficiently used for irradiating microorganisms. UV radiation that did not interfere with a microorganism the first time it passed the UV treatment chamber may interfere with another microorganism after it has been reflected by the reflecting layer on the inner wall of the UV treatment chamber.

It has been found that the metal lattice of aluminum is specifically suitable for constructing the reflective layer. The wavelengths of the UV radiation that is used are at least partially reflected by aluminum.

To fill the UV treatment chamber with UV radiation coming from all possible directions and thus increasing the chance of interference with passing microorganisms, it is advantageous to scatter the UV radiation, when it is reflected. Therefore, it is advantageous that the reflective layer has a rough surface such that reflected UV radiation is scattered. In a specific embodiment, the reflective layer is formed by sputtered aluminum, since such a sputtered layer of aluminum reflects and scatters the incident UV radiation.

In an advantageous embodiment, the air treatment device further comprises a cooling unit upstream relative to the UV treatment chamber for cooling and/or dehydrating the airflow.

The cooling unit, which may receive air only containing small particles, which are mainly bacteria, viruses, fungi and other microorganisms, has two functions. The cooling unit cools the air, and it dehydrates the air. The air is cooled to provide air with an optimal temperature to the UV treatment filter. Which temperature is optimal will be described hereinafter.

The air is dehydrated to prevent that water molecules become attached to the microorganisms, since attached water molecules form a shield against UV radiation around the microorganisms. It has been found that it may take up to a four times higher dose of UV radiation to kill a microorganism having a water molecule shield around it. Dehydrating the air results in less shielding and thus results in requiring less UV radiation in the UV treatment filter to kill bacteria.

Dehydration is established by cooling the air. Cold air can contain less water molecules than hot air. Cooling the air results in condensation of a percentage of the water present in the air. The condensed water may be stored in a tank, which is to be emptied by a person when it is full. Also, the condensed water may be directly drained. In a specific embodiment, the condensed water may be vaporized in the airflow again after the microorganisms have been killed to prevent that unnaturally dry air is output by the air treatment device.

In an advantageous embodiment, the air treatment device comprises an ionizer, downstream relative to said at least one filter if present, and downstream to said cooling unit if present, for providing an electron stream substantially perpendicular to the direction of airflow.

The ionizer generates an electrical field. A function of the ionizer results from an electron stream inevitably running from one pole of the ionizer to the other. Microorganisms may get hit by one or more electrons and get killed or weakened. If the ionizer is positioned downstream to the UV treatment chamber, any microorganisms, which inadvertently have been able to survive the UV treatment filter, possibly having been mutated, get irrigated with the electrons in said stream and get killed. To provide a large electron stream, the poles of the ionizer may be designed with a large surface. For example, the poles may be constructed as a brush of electrically conducting wires.

The ionizer may further function to re-hydrate the passing air. As an electrical field is generated between two electrical poles of the ionizer, water molecules get polarized, i.e. they orientate themselves all in a same direction. This is an effect that is well known to a person skilled in the art. Due to the polarization, the water molecules become easily attached to molecules in the air, hydrating the air to a natural hydration level.

In an embodiment of the device according to the present invention, the air treatment device further comprises a second carbon filter downstream relative to the filter. A carbon filter is known in the art for capturing gases, and thus reducing smells present in the airflow.

In an even further embodiment, the cooling unit and the carbon filter may be combined in one filter. The combined filter may capture liquids, in particular water, and gases by polarization and cool the air. By controlling an electrical potential of electrodes comprised in the combined unit the humidity and the temperature of the air passing the combined filter may be controlled.

To control the humidity, and thus the amount of water adhering to microorganisms, the air treatment device may comprise a humidity sensor downstream relative to the cooling unit, which sensor determines the humidity of the air and outputs corresponding humidity data. The humidity data are received by a processing device from the humidity sensor, which processing device controls the cooling unit to provide a predetermined humidity in the UV treatment chamber. Thus, the humidity of the air in the UV treatment chamber may be kept at the predetermined humidity level irrespective of the humidity of the air entering the air treatment device at the air inlet. Preferably, the humidity sensor is disposed in the UV treatment chamber to obtain the humidity level in the UV treatment chamber directly.

Similarly, to control the temperature, the air treatment device may comprise a temperature sensor downstream relative to the cooling unit, which sensor determines the temperature of the air and outputs corresponding temperature data. The temperature data are received by a processing device from the temperature sensor, which processing device controls the cooling unit to provide a predetermined temperature in the UV treatment chamber of the UV treatment filter. Thus, the temperature of the air in the UV treatment chamber may be kept at the predetermined temperature level as long as the temperature of the air entering the air treatment device at the air inlet is higher than the predetermined temperature.

In an embodiment of the air treatment device, the first temperature sensor is disposed immediately downstream of the UV treatment chamber. The temperature of the air leaving the UV treatment chamber is a measure for the amount of UV radiation being radiated on the microorganisms. Thus, by determining and controlling the temperature of the outgoing air, it may be ensured that the microorganisms have received enough UV radiation to be killed.

In an embodiment, the at least one UV radiation source may be provided with a second temperature sensor and a processing device receives temperature data from said second temperature sensor. The processing device controls a power output of the UV radiation source based on the received temperature data to protect the UV radiation source from undercooling or overheating. Since the temperature of the air flowing into the UV treatment chamber may vary and since the airflow rate into the UV treatment chamber may vary, the second UV radiation source may have a problem of creating or exchanging heat generated during operation, which may result in overheating or undercooling. Overheating or undercooling is prevented by determining the temperature of the UV radiation source and adjusting the output power of the UV radiation source based on said determined temperature.

Advantageously, the first and/or second UV radiation source is disposed in a cover, which cover is transmissive for the emitted UV radiation. The cover protects humans against harmful chemical compounds present in the UV radiation source, if the UV radiation source should break. Further, such a cover may protect in particular the UV radiation source against abrupt cooling down due to cold air entering the air treatment device. This is specifically advantageous, because cold air entering the UV treatment chamber adversely influences the air treatment capacity of the UV treatment chamber. A suitable cover is made of Teflon, since Teflon is transmissive for the used UV radiation and Teflon does not degrade in course of time due to the light.

It is noted that a cover transmissive for the emitted light of a light source may as well be advantageously employed in combination with any other light source comprising harmful chemical compounds, for example tube lights (TL) and gas discharge lamps, in order to contain said chemical compounds in case of breakage of the light source. Also, in combination with lamps constructed of glass, a transmissive cover may be employed to contain shattered glass splinters in case of breakage.

The air inlet and the air outlet of the housing of the air treatment device may be constructed such that no UV radiation may escape from the housing, since the used UV radiation is harmful to humans. A person skilled in the art readily understands how such a construction may be designed. For example, a maze-like construction may be used. Further, an UV radiation absorbing layer may be provided on a wall of the housing, or part thereof.

The air treatment device according to the present invention can be used in medical, residential, commercial, industrial and military and animal growing applications, either as a stand-alone unit, or as part of a further air conditioning system.

In another aspect, the present invention provides an air treatment method comprising generating an airflow; and radiating UV radiation for exposing said airflow to said UV radiation for killing a microorganism present in said airflow.

Aspects, advantages and features of the device according to the invention are explained in more detail by reference to the accompanying drawings illustrating exemplary embodiments, in which:

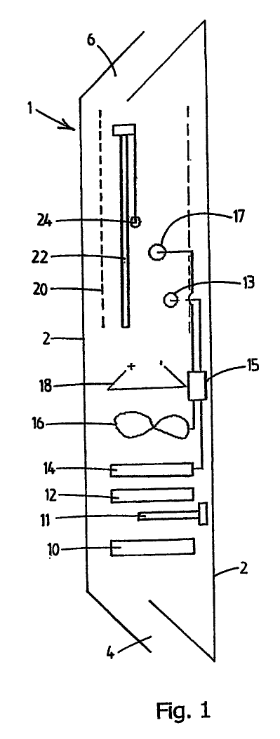

FIG. 1 schematically shows the structure of an air treatment device according to the present invention;

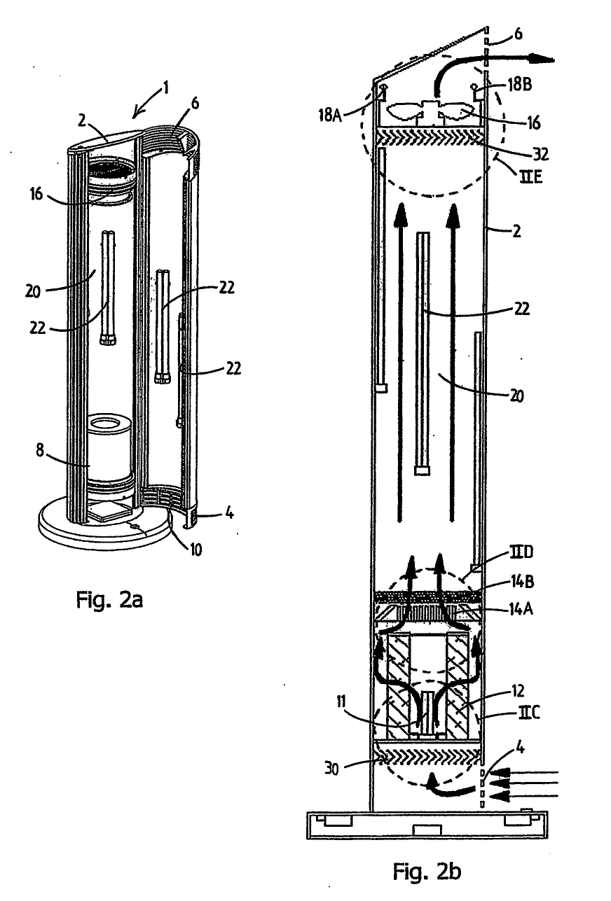

FIG. 2A shows a perspective view of an air treatment device according to an embodiment of the present invention;

FIG. 2B shows a sectional view of the embodiment illustrated in FIG. 2A;

FIGS. 2C-2E show parts of the sectional view of FIG. 2B on a larger scale;

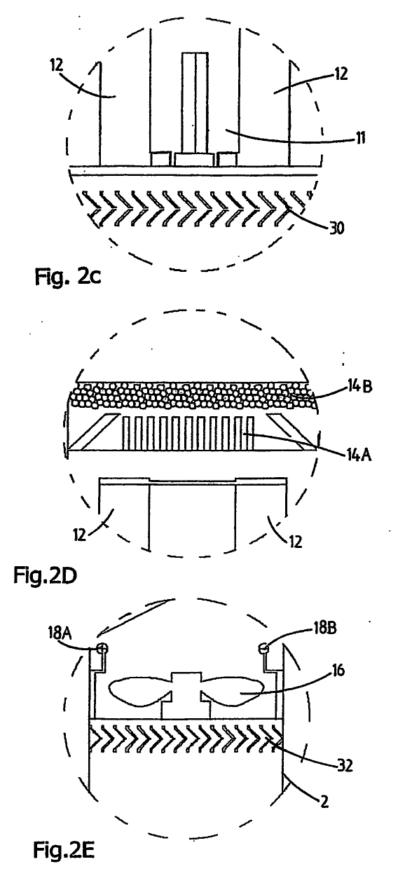

FIG. 3 shows a graph of a pollutant removal factor as a function of a pollutant size; and

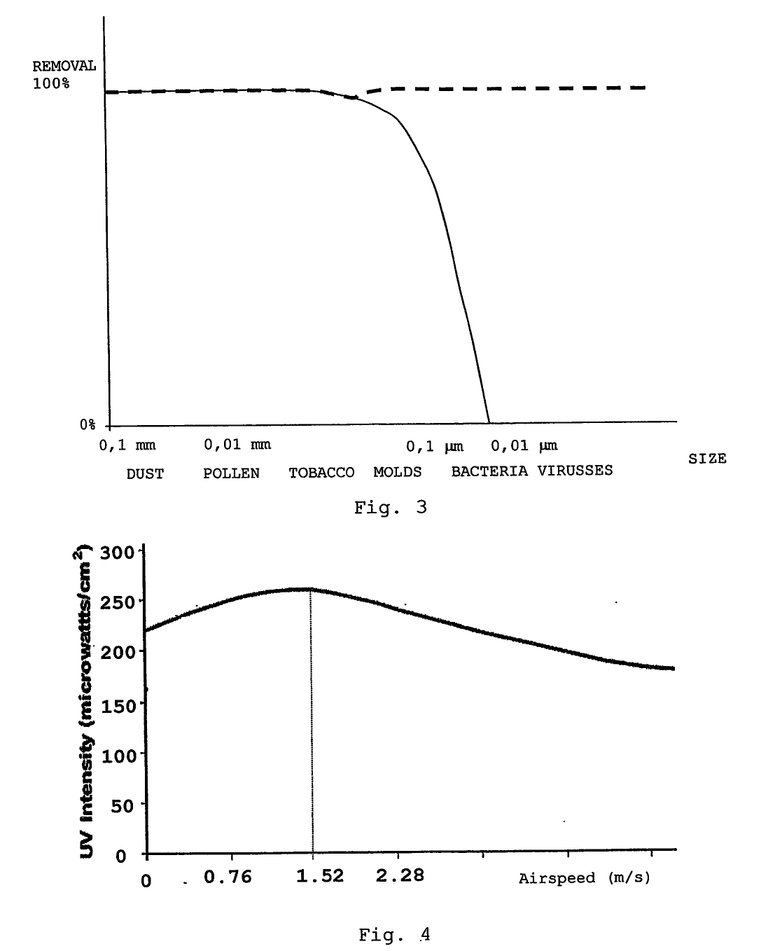

FIG. 4 shows a graph of a UV radiation source efficiency as a function a cooling air flow rate.

In the different Figures, like reference numerals indicate like components or components having the same function.

FIG. 1 schematically illustrates the arrangement of various components in an air treatment device, which is generally indicated with reference numeral 1.

The air treatment device 1 comprises an elongated tube-like enclosure 2, having a cross-section which is generally circular or oval shaped, or has any other suitable cross-sectional shape, such as a rectangular or multiangular shape. The shape or the area of the cross-section of the enclosure 2 may vary along its length. In a preferred embodiment, the cross-section is circular, is constant along the length of the enclosure 2, and has a diameter of about 0.2-0.3 meters.

The enclosure has an air inlet 4 at a first end thereof, and an air outlet 6 at a second end thereof. Air generally is intended to flow through the enclosure 2 from the air inlet 4 to the air outlet 6. In one embodiment, a longitudinal axis of the enclosure 2 may be directed upright or generally vertically, with the air inlet 4 located at the lower end of the enclosure 2, and the air outlet 6 located at the upper end of the enclosure 2. However, in principle any orientation of the air treatment device may be selected.

From the air inlet 4 to the air outlet 6, air flowing through the enclosure 2 follows a path through or along various components, such as a dust filter 10, a HEPA filter 12, a carbon filter 14, a fan 16, an ionizer 18, and a UV treatment chamber 20 containing at least one UV radiation source 22, in order to ensure the capture of particles and/or the termination of substantially all viruses, bacteria and other harmful microorganisms in the air treatment device. Although the dust filter 10, the HEPA filter 12, and the carbon filter 14 are shown in FIG. 1 to be free from the enclosure 2, in a practical embodiment they extend to an inner wall (indicated with dashed lines) of the enclosure 2 to ensure that all air flowing through the enclosure 2 passes through each of these filters.

The dust filter 10 is situated downstream relative to the air inlet 4 to capture dust particles in the air having relatively large dimensions. The dust filter 10, being the first filter in the air treatment device 1, is also referred to as a prefilter. Preferably, the dust filter 10 is exchangeable and/or washable.

The HEPA (High Efficiency Particulate Air) filter 12, preferably manufactured from microfiberglass, is situated downstream relative to the dust filter 10, to capture small particles with sizes of about 0.1 to 0.3 microns and higher. The HEPA filter 12 may remove as much as 99.97% of airborne pollutants, and will further capture at least part of the total amount of viruses, bacteria, and fungae present in the air. A relatively small UVC (Ultra Violet rays type C) radiation source 11 situated in the vicinity of the HEPA filter 12 will kill the viruses, bacteria, and fungae captured in the HEPA filter 12 in the course of time. Preferably, the HEPA filter 12 is exchangeable. Also preferably, the UVC radiation source 11 emits radiation at about 253 nanometres or any other suitable wavelength, and at an operating temperature of 40° C. or any other suitable operating temperature. The UVC radiation source 11 is preferably placed at the side of the HEPA filter 12 facing the air inlet 4 of the enclosure 2.

The carbon filter 14 is situated downstream relative to the HEPA filter 12, and comprises electrodes (not shown) with an adjustable potential, to capture liquids (in particular water) and gases by polarization. Thus, the humidity of the air passing the carbon filter 14 may be controlled by controlling the potential of the electrodes of the carbon filter 14. By controlling the humidity of the air, the amount of water adhering to viruses and bacteria may be controlled with a view to controlling the effectiveness of the air treatment in the UV treatment chamber 20. A humidity sensor 13 located downstream relative to the carbon filter, preferably located in the UV treatment chamber 20, provides humidity data which are processed in a processing device 15 coupled to the humidity sensor 13. The processing device 15 is coupled to the electrodes of the carbon filter 14, and controls the potential of the electrodes in a predetermined manner such as to achieve a predetermined humidity of about 40-50% in the UV treatment chamber 20, irrespective of the humidity of the air entering the air inlet 4 of the air treatment device 1. Gases are also captured in the carbon filter 14, thus reducing any smells present in the air flowing through the air treatment device 1.

The fan 16 is situated downstream relative to the carbon filter 14 to generate high air flows in the air treatment device 1. A temperature sensor 17 is located in the UV treatment chamber 20, and coupled to a processing device (which may or may not be the same as the processing device 15 described above). The processing device is coupled to a motor of the fan 16, and controls the motor speed (and thus the flow rate of the air in the air treatment device 1) for achieving a predetermined temperature in the UV treatment chamber 20. This temperature depends on the amount of cooling of the at least one UVC radiation source 22 in the UV treatment chamber 20 by the air flowing by the at least one UVC radiation source 22.

In a practical embodiment, typically the air should flow along the at least one UVC radiation source 22 with a speed of about 1.5 meters/second to reach a steady state temperature in the UV treatment chamber 20 of about 40° C. Such a temperature will effect an optimum sterilization of the air in the UV treatment chamber, which can be achieved irrespective of the air temperature of the air entering the air treatment device at the air inlet 4, by controlling the motor speed of the fan 16. Depending on the configuration of the air treatment device 1, airflow delivery rates of 76 cubic meters per hour up to 380 cubic meters per hour (hyper dynamic flows) are possible, which would lead to an average room with a floor area of 4×8 metres having its entire volume treated in the air treatment device 1 several times per hour. It is noted that a minimum airflow rate of approximately 1.5 meters/second is needed to ensure that an airflow is generated in the whole room such that substantially all air present in the room may be treated.

By placing the fan 16 downstream relative to the dust filter 10, the HEPA filter 12, and the carbon filter 14, the fan 16 can be kept clean. However, if the fan 16 would be positioned upstream to one or more of said filters and it would get polluted, any filter downstream to the fan 16 will remove any particle airborne from said polluted fan 16.

The ionizer 18 is located downstream relative to the fan 16, and returns the ionization of the air to natural, human-friendly values.

The UV treatment chamber 20 contains the at least one UVC radiation source 22, preferably emitting UVC radiation at about 253 nanometres or any other suitable wavelength, and preferably being driven at 100% power output, when operating at 40° C. The at least one UVC radiation source 22 has an integrated temperature sensor 24 protecting the at least one UVC radiation source 22 from undercooling or overheating by adapting the power output thereof accordingly. The walls of the UV treatment chamber 20 are manufactured to provide a maximum reflection of UVC radiation. For this purpose, preferably aluminum has been sputtered on the walls of the UV treatment chamber 20. Accordingly, direct and up to 7 times reflected UVC radiation may increase the sterilizing efficiency of the UV treatment chamber 20 by 300%. The at least one UVC radiation source 22 is constructed such, that no ozone is created by its operation.

The air outlet 6 is constructed such that no UVC radiation may escape from the air treatment device 1. A special radiation absorbing paint is applied to the walls of the air outlet 6, and a maze-like structure of the air outlet 6 prevents any radiation from leaving the device.

The signals generated by the temperature sensors 17 and 24, and the humidity sensor 13 are evaluated in respective processing devices coupled thereto, and the processing devices are adapted to turn off the air treatment device 1 if a potentially abnormal situation is detected, or if a situation arises in which a condition for replacement of a component of the air treatment device 1 is met. Examples of such situations are: stopping of the fan 16, overheating or undercooling of components, in particular the at least one UVC radiation source 22, exchange period of filter reached, etc.

FIG. 2A shows an enclosure 2 with a circular cross-section. A front side of said enclosure 2 has been hinged away to expose the components accommodated in the enclosure 2. Said front side comprises the air inlet 4 and the air outlet 6. At the inside of the air inlet 4, the dust filter 10 is provided.

The air treatment device 1 further comprises a filter enclosure 8, comprising a HEPA filter, a first UV radiation source and possibly a cooling unit and/or a carbon filter. In the embodiment illustrated in FIG. 2A, the UV treatment chamber is provided with four UV radiation sources 22 to provide enough UV radiation per unit time to kill all microorganisms passing through the UV treatment chamber per unit time. The fan 16 is disposed immediately upstream to the air outlet 6.

FIG. 2B shows a sectional view of the elements present in the air treatment device 1 of FIG. 2A. The arrows in FIG. 2B indicate the direction of airflow through the air treatment device 1.

The air inlet 4 and the air outlet 6 are provided at two ends of the enclosure 2. A first UV protective cover 30 is provided between the UV radiation sources and the air inlet 4. Similarly, a second UV radiation protective cover 32 is provided upstream to the air outlet 6. Said first and second protective covers 30 and 32 ensure that no UV radiation may pass and leave the air treatment device 1. Air flowing through the treatment device 1 may freely pass through the protective covers 30 and 32.

In FIG. 2C, which is an enlarged part of FIG. 2B, as indicated in FIG. 2B with IIC, the construction of the UV protective cover 30 is illustrated on a larger scale. Using V-shaped plates, preferably coated with an UV radiation absorbing layer, and positioned as shown, prohibits UV radiation passing, but an air flow may freely pass.

Referring to FIG. 2B again, the HEPA filter 12 is cylindrically shaped and coaxially disposed in the enclosure 2, thus providing a large filter surface. The large filter surface provides a low airflow resistance and good filter characteristics, such as long use life and high filter capacity. The first UV radiation source 11 is disposed in a center of the HEPA filter, as also may be seen in FIG. 2C, radiating its UV radiation on the surface of the HEPA filter around it. Such a configuration has a further advantage that a direction of the UV radiation is substantially perpendicular to a surface of the HEPA filter. Thus, the UV radiation is more efficiently used, since there are no spots or fibers on the HEPA filter that may be shielded by other fibers.

In the illustrated embodiment, as also may be seen in FIG. 2D (IID in FIG. 2B), also a cooling unit 14A and a carbon filter 14B are provided in the filter enclosure 8. Further, the four UV radiation sources 22 disposed in the UV treatment chamber 20 are positioned relative to each other such that in operation the UV radiation intensity inside the UV treatment chamber 20 is substantially homogenous.

As shown in FIGS. 2B and 2E (indicated as IIE in FIG. 2B), downstream to the UV treatment chamber 20, the second UV protective cover 32 is disposed, and further downstream a fan 16 and an ionizer comprising a positive pole 18A and a negative pole 18B are provided.

It is noted that the embodiment of the air treatment device 1 illustrated in FIGS. 2A-2E may comprise a number of sensors, such as one or more temperature sensors, one or more humidity sensors, and/or microorganism sensors, although they are not shown in FIGS. 2A-2E. Further, the embodiment illustrated in FIGS. 2A-2E functions substantially similar to the embodiment of FIG. 1.

Said microorganism sensors may determine a number of microorganisms present in the air. Such a sensor may be provided immediately downstream to the air inlet 4 and immediately upstream to the air outlet 6. Coupling said microorganism sensors to a processing device enables to determine a sterilization factor or the like. Such a sterilization factor may be displayed. In a more sophisticated embodiment, the number of microorganisms present in the air may as well be used to control the air treatment device 1.

Since the air treatment device according to the present invention employs UV radiation of a possibly harmful wavelength, an embodiment may be provided with a number of security measures, such as an opening sensor, which detects opening of an enclosure and may shut down any UV radiation source to prevent UV radiation radiating on any person.

Further, the UV radiation sources may be of a kind that does not generate ozone and the air treatment device may as mentioned above be provided with a display for informing any user of the status of the air treatment device and/or any of the filters. The display may be connected to a processing device that also controls the air treatment device.

As mentioned above, the method and device according to the present invention are suited for killing substantially all microorganisms present in airflow having a high airflow rate, whereas prior art air treatment devices only filter relatively large microorganisms and dust particles from an air flow. FIG. 3 shows a graph illustrating a microorganism removal rate as a function of a size of the microorganisms. The microorganisms are classified into a number of groups depending on their size: dust, pollen, tobacco (smoke), molds, bacteria and viruses. The solid line represents a performance of a prior art air treatment device and the dashed line represents a performance of the air treatment device according to the present invention.

The prior art device removes up to 100% of all pollutants having a size of up to 1 micrometer. Some smaller pollutants are removed, but pollutants smaller than about 0.1 micrometer remain in the air. Thus, up to about 99.97% of the pollutants may be removed from the air. Since sterilization is defined as removing at least 99.9999% of the pollutants, the prior art air treatment device may be indicated to be an air purifier.

The air treatment device according to the present invention also removes smaller air pollutants from the air. As shown by the dashed line, up to 100% of all pollutants are removed. Tests of independent laboratories (Microsearch Laboratories Ltd. (United Kingdom) and Biotec (Germany)) have shown that more than 99.9999% of the pollutants are removed by the air treatment device according to the present invention. Thus, according to the above-mentioned definition of sterilization, the air treatment device according to the present invention may be indicated to be an air sterilizer.

To prevent that mutated organisms may leave the air treatment device, all microorganisms need to be killed. Therefore, each microorganism being exposed to UV radiation is to receive a minimum dose of UV radiation that kills said microorganism. A number of measures may be taken to increase the efficiency of the UV radiation source and the UV radiation output by said UV radiation source. For example, the UV treatment chamber may be provided with a reflective layer, the air may be prefiltered, the air may be dehydrated, and the air temperature and airflow rate may be controlled.

FIG. 4 illustrates the output efficiency of an UV radiation source as a function of an airflow rate of an airflow passing the UV radiation source, the air having a temperature of about 20° C. An UV radiation output of the UV radiation source is dependent on the operating temperature. An optimal operating temperature of the UV radiation source is 40° C. as mentioned above. Due to the passing air, the UV radiation source is cooled. If airflow cools the UV radiation source, the power consumption may be increased above a rated power level to increase the heat generation. Thus, the radiation source may be kept at its optimal operating temperature.

As illustrated in FIG. 4, the UV radiation source is efficiently driven in airflow having an airflow rate of about 1.52 meters/second (about 300 feet per minute), which is higher than a minimum required airflow rate of 1.5 meters/second as discussed above. At the same time, the UV radiation source is driven at a power higher than a rated power, thereby generating heat to substantially compensate the cooling effect of the passing air. It is noted that a suitable cover over the UV radiation source as mentioned above may prevent the UV radiation source from abrupt cooling.

The air treatment method according to the present invention, which is practically embodied in the air treatment device according to the present invention, may as well be employed in other treatment devices. For example, for sterilizing objects, UV-C treatment may be very suitable. In hospitals, for example, many objects need to be sterilized. Further, instead of air, other fluids may be sterilized, such as gases, e.g. oxygen used in hospitals, and water. Depending on the application, prefiltering may be employed.

With the air treatment device and method according to the present invention, bounded spaces can be safely decontaminated, in particular by killing all viruses, bacteria, fungae and other potentially harmful microorganisms, and by removing dust and other particles. The design of the air treatment device is based on an UV dose required to kill any microorganism. A number of parameters, e.g. the measures of the UV treatment chamber, the airspeed inside the UV treatment chamber and the air outlet speed of the airflow, as described in detail above, are selected such that substantially all microorganisms in a dynamic airflow are killed, while it is ensured that cleaned air mixes with the air present in a room. This means that air on another side of the room is forced to the inlet of the air treatment device. Thus, it is prevented that a number of microorganisms may mutate into harmfull microorganisms.

1-38. (canceled)

39. An air treatment device comprising:
a housing including an air inlet and an air outlet;
a fan for stimulating an airflow through the housing from the air inlet to the air outlet; and
a UV treatment chamber downstream relative to said air inlet, the UV treatment chamber including at least one UV radiation source for exposing said airflow to UV radiation for killing a microorganism present in said airflow.

40. The air treatment device according to claim 39, further comprising at least one filter upstream relative to the UV treatment chamber for removing particles and microorganisms having a size larger than a predetermined filter diameter from said airflow before exposing said airflow to said UV radiation.

41. The air treatment device according to claim 40, further comprising:
a dust filter downstream relative to the air inlet for removing large dust particles from said airflow; and
a HEPA filter downstream relative to the dust filter for removing small dust particles and large microorganisms from the airflow.

42. The air treatment device according to claim 40, further comprising a carbon filter downstream relative to the air inlet for removing dust particles and microorganisms from said airflow.

43. The air treatment device according to claim 40, wherein a filter UV radiation source is provided for irradiating UV radiation on at least one of said at least one filter.

44. The air treatment device according to claim 39, wherein the fan is positioned upstream relative to the UV treatment chamber such that the airflow in the UV treatment chamber is substantially turbulent.

45. The air treatment device according to claim 40, further comprising a cooling unit downstream relative to said at least one filter for cooling, and dehydrating by cooling, the airflow.

46. The air treatment device according to claim 45, further comprising a humidity sensor disposed downstream relative to the cooling unit, and a processing device which receives humidity data from said humidity sensor, with the processing device controlling the cooling unit to provide a predetermined humidity in the UV treatment chamber.

47. The air treatment device according to claim 46, wherein the humidity sensor is disposed in the UV treatment chamber.

48. The air treatment device according to claim 45, further comprising a first temperature sensor disposed downstream relative to the cooling unit, and a processing device which received first temperature data from said first temperature sensor, with the processing device controlling the airflow rate by controlling a fan speed, to provide a predetermined temperature of the air leaving the UV treatment chamber.

49. The air treatment device according to claim 48, wherein the temperature sensor is disposed immediately downstream relative to the UV treatment chamber.

50. The air treatment device according to claim 39, further comprising an ionizer, which is located downstream relative to said at least one filter, for providing an electron stream substantially perpendicular to the direction of airflow.

51. The air treatment device according to claim 45, further comprising an ionizer, which is located downstream relative to the cooling unit, for providing an electron stream substantially perpendicular to the direction of airflow.

52. The air treatment device according to claim 39, further comprising a second carbon filter located downstream relative to said at least one filter.

53. The air treatment device according to claim 45, further comprising a second carbon filter downstream relative to said at least one filter, the carbon filter and the cooling unit being combined in one unit.

54. The air treatment device according to claim 39, wherein an inner wall of the UV treatment chamber is provided with a UV radiation reflecting layer.

55. The air treatment device according to claim 54, wherein the reflecting layer consists of aluminum.

56. The air treatment device according to claim 54, wherein the reflecting layer has a rough surface such that reflected UV radiation is scattered.

57. The air treatment device according to claim 54, wherein the reflecting layer is formed by sputtered aluminum.

58. The air treatment device according to claim 39, further including a second UV radiation source provided with a second temperature sensor and a processing device which receives second temperature data from said second temperature sensor, said processing device controlling a power output of said at least one UV radiation source for protecting the at least one UV radiation source from undercooling or overheating.

59. The air treatment device according to claim 39, further comprising at least one microorganism sensor for determining a number of microorganisms present in the air passing said microorganism sensor.

60. The air treatment device according to claim 59, wherein said microorganism sensor is connected to a processing device, the processing device controlling the air treatment device in response to the determined number of microorganisms.

61. The air treatment device according to claim 59, comprising a first microorganism sensor provided immediately downstream of the air inlet and a second microorganism sensor provided immediately upstream to the air outlet, with said first and said second microorganism sensors connected to a processing device, the processing device determining a sterilization factor from a determined number of microorganisms present in the air flowing into the air treatment device and a determined number of microorganisms present in the air flowing out of the air treatment device.

62. The air treatment device according to claim 39, wherein the at least one UV radiation source is disposed in a cover, which cover is transmissive for the emitted UV radiation.

63. The air treatment device according to claim 62, wherein the cover is made of Teflon.

64. The air treatment device according to claim 39, wherein the air inlet and the air outlet in the housing are constructed such that no UV radiation may escape from the housing.

65. The air treatment device according to claim 39, wherein an UV radiation absorbing layer is provided on a wall of the housing.

66. The air treatment device according to claim 39, wherein the emitted UV radiation of said at least one UV radiation source has a wavelength between 253 nm and 257 nm, preferably a wavelength of 253.7 nm.

67. The air treatment device according to claim 43, wherein the emitted UV radiation of the filter UV radiation source has a wavelength between 253 nm and 257 nm, preferably a wavelength of 253.7 nm.

68. An air conditioning system comprising an air treatment device, the air treatment device comprising:
a housing comprising an air inlet and an air outlet;
a fan for stimulating an airflow through the housing from the air inlet to the air outlet;
a dust filter downstream relative to the air inlet for removing large dust particles from said airflow;
a HEPA filter downstream relative to the dust filter for removing small dust particles and large microorganisms from the airflow;
a first UV radiation source for irradiating UV radiation on the HEPA filter; and
an UV treatment chamber downstream relative to said HEPA filter, the UV treatment chamber comprising a second UV radiation source for irradiating UV radiation in said UV treatment chamber.

69. An air treatment method comprising the steps of:
(a) generating an airflow; and
(b) radiating UV radiation for exposing said airflow to said UV radiation for killing a microorganism present in said airflow.

70. The air treatment method according to claim 69, further comprising filtering particles and microorganisms having a size larger than a predetermined filter diameter from said airflow before exposing said airflow to said UV radiation.

71. The air treatment method according to claim 69, further comprising dehydrating the airflow before exposing said airflow to said UV radiation.

72. The air treatment method according to claim 69, further comprising the steps of:
determining an air temperature of said airflow; and
controlling an airflow rate in response to said air temperature.

73. The air treatment method according to claim 69, further comprising generating an electron stream in said airflow, the electron stream being substantially perpendicular to the direction of said airflow.

74. The air treatment method according to claim 69, further comprising:
determining a temperature of a UV radiation source; and
controlling a power consumption of said UV radiation source for protecting said UV radiation source against overheating or undercooling.

75. The air treatment method according to claim 69, further comprising the steps of:
determining a number of microorganisms present in said airflow; and
controlling at least one of an airflow rate, hydration level and a radiation source power consumption in response to the determined number of microorganisms.

76. The air treatment method according to claim 75, further comprising the steps of:
determining an input number of microorganisms present in said airflow before exposing said airflow to said UV radiation;
determining an output number of microorganisms present in said airflow after exposing said airflow to said UV radiation; and
determining a sterilization factor from said input number of microorganisms and said output number of microorganisms;
wherein said at least one of an airflow rate, hydration level and a radiation source power consumption is controlled in response to said sterilization factor.
